(19) United States
(12) Patent Application Publication
(10) Pub. No.: US 2025/0302307 A1
(43) Pub. Date: Oct. 2, 2025

(54) MEDICAL IMAGING DEVICE AND METHOD SUITABLE FOR OBSERVING A PLURALITY OF SPECTRAL BANDS

(71) Applicant: FLUOPTICS, GRENOBLE (FR)

(72) Inventors: PHILIPPE RIZO, LA TRONCHE (FR); MARIEKE RICHARD, SASSENAGE (FR)

(21) Appl. No.: 18/863,972

(22) PCT Filed: Feb. 17, 2023
(86) PCT No.: PCT/EP2023/054072

§ 371 Date: Nov. 7, 2024

(30) Foreign Application Priority Data
May 19, 2022 (FR) FR2204792

(51) Int. Cl. A61B 5/00

(52) U.S. Cl. CPC A61B 5/0071; A61B 5/0082; A61B 5/415

(57) ABSTRACT

The invention relates to a fluorescence imaging method and device (I) for surgical applications. Filtering means (10) are used that are configured to be able to generate, from the output signal, at least two images each corresponding to a different filtering operating mode over at least one wavelength range. The respective contribution to the intensity of the output signal obtained at a first wavelength and at a second wavelength, which are distinct from one another, is different with each of these two filtering operating modes. The filtering operating modes are chosen so as to highlight, in an image, the fluorescence emission of one substance relative to the fluorescence emission of another substance that might be present in a region of interest.

### TECHNICAL AREA

The invention relates to the field of medical imaging and, in particular, fluorescence imaging.

### PRIOR ART

In medicine, fluorescence imaging consists of injecting a fluorescent marker, which, excited at certain wavelengths by a light source, generates fluorescence radiation that can be captured by a camera and visualized in real time (this is called exogenous fluorescence).

Some human tissues intrinsically emit (without added fluorescent label) fluorescent radiation when excited at certain wavelengths; this is called tissue autofluorescence (also called endogenous fluorescence)

The major advantage of autofluorescence imaging is that it is completely non-invasive for the patient (no intravenous or subcutaneous tracer injection).

However, autofluorescence imaging and fluorescence imaging can provide additional information useful for clinical interpretation.

For example, during a thyroidectomy, the thyroid gland must be removed while preserving the parathyroid glands, which are generally very close to it. Locating the parathyroid glands is often difficult with the naked eye and requires a great deal of experience on the part of the surgeon. The autofluorescence of these glands facilitates their localization.

Thus, some surgeons have been able to demonstrate that the number of post-operative complications decreases when the surgeon secures his procedure using autofluorescence imaging.

However, autofluorescence imaging only provides contextual information, namely, “Where are the parathyroid glands”. But, during thyroid dissection, the surgeon also needs functional information: 1) identify the vessels that irrigate each of these parathyroid glands so as not to damage them and preserve their functionality. 2) verify that the parathyroid glands are well vascularized. Autofluorescence imaging alone does not allow observation of the irrigating vessels or tissue perfusion. On the other hand, it is conventional to use the fluorescence of a marker such as “Indocyanine Green” or ICG to visualize the vascular network that supplies the parathyroid glands.

Now, a thyroidectomy is performed in two stages. The two lobes of the thyroid are resected successively.

With prior art devices and methods, when dissecting the first lobe, only autofluorescence can be used to visualize the parathyroid glands. In fact, the injection of indocyanine green can only be used after having visualized the parathyroid glands on the second side by autofluorescence. In fact, with current systems that also measure autofluorescence, it is no longer possible to visualize the autofluorescence of the parathyroid glands after an injection of indocyanine green. With the excitation wavelength and detection wavelength band commonly used by systems for measuring autofluorescence and indocyanine green fluorescence, after injection, indocyanine green fluorescence masks the parathyroid autofluorescence. Under these conditions, in order to visualize the autofluorescence of the parathyroid glands after injection of indocyanine green, it would be necessary to wait for a period incompatible with the duration of the surgery itself (between half an hour and an hour) for the concentration of indocyanine green to be sufficiently low. But even after such a long waiting time, as the injection of indocyanine green is carried out on tissues undergoing dissection, many areas remain marked by the indocyanine green that is not evacuated, and therefore, risk degrading the quality of the interpretation of the autofluorescence images by adding a large number of false positives. The injection of indocyanine green is therefore reserved for a stage of the thyroidectomy which no longer requires observation of the autofluorescence of the parathyroid glands. Perfusion imaging can, therefore, only be used on the second side of the operation, at the end of the procedure.

However, the main disadvantage of not using perfusion imaging on the first side leads to (i) an increased risk of damaging the vascularization of the parathyroid glands of the first lobe, (ii) not knowing whether the parathyroid glands left in place are functional, which is of crucial importance on how to operate on the second lobe, especially if in this second lobe the parathyroid glands are difficult to protect.

It is, therefore of prime importance to be able to visualize, in situ, the autofluorescence of the parathyroid glands even after the injection of indocyanine green.

Ideally, for each lobe, autofluorescence imaging of the parathyroid glands, fluorescence visualization of the vessels (fluorescence angiography) during dissection, and validation of parathyroid gland perfusion at the end of the lobectomy should be performed.

Therefore, there is a need to visualize autofluorescence alone (or almost) even after the injection of indocyanine green. But more generally, there is a need to separately visualize fluorescent substances whose fluorescence emission exhibits sufficiently distinct emission spectra.

In this document, a fluorescent substance may consist of one molecule or several molecules.

### DISCLOSURE OF THE INVENTION

The invention aims to provide at least a partial response to the aforementioned need. For this purpose, a fluorescence imaging method for surgical applications is provided comprising an operation of exciting at least a first and a second fluorescent substance potentially located in an area of interest, with at least one excitation radiation having a maximum intensity defined by an excitation wavelength, this excitation wavelength being between 600 nm and 800 nm, or more particularly between 650-750 nm. The first substance has a fluorescence emission spectrum with a first intensity maximum at a first wavelength (e.g., indocyanine green fluorescence with an intensity maximum near 820 nm), and the second substance has a fluorescence emission spectrum with a second intensity maximum at a second wavelength (e.g., parathyroid gland autofluorescence with an intensity maximum near 711 nm). The fluorescence spectra of the first and second substances are different. The first and second wavelengths are different and dependent on the excitation wavelength. For example, the fluorescence spectrum maximum of the first substance has a wavelength greater than the fluorescence spectrum maximum of the second substance, with the excitation wavelength used to excite the fluorescence in the area of interest.

The method, according to the invention, further comprises
an operation for filtering at least a portion of the fluorescence radiation emitted by each fluorescent substance emitting fluorescence radiation in response to excitation radiation in the zone of interest and
an operation of detection and acquisition, by means of a sensor, of at least a portion of the filtered fluorescence radiation, this detection operation being carried out in a detection range itself included in a wavelength range between 400 nm and 1000 nm (i.e., the conventional sensitivity range of imaging CMOS sensors), possibly, for example, this detection length range extends between the excitation wavelength and 900 nm or 1000 nm, or even between the excitation wavelength and 870 nm, in other words, the detection operation can be performed over a wavelength range that extends between a first wavelength greater than or equal to 400 nm and a second wavelength less than or equal to 1000 nm,
an operation consisting of generating, from the radiation detected by the sensor, at least one output signal whose intensity is representative of the intensity of the fluorescence radiation detected by the sensor,
an operation for displaying images formed from the output signal.

Furthermore, in this method, the filtering operation is carried out according to at least two different operating procedures so as to generate at least two images each respectively from the output signal, using one of these different operating procedures, implemented one after the other. The respective contribution to the intensity of the output signal obtained at the first and second wavelength levels is different with each of these two operating procedures. For example, with one of the two operating procedures, the contribution to the output signal intensity obtained at the level of the second wavelength is greater than or equal to that obtained at the level of the first wavelength. In the other operating procedure, the opposite is true. The contribution to the output signal intensity obtained at the level of the first wavelength is greater than or equal to that obtained at the level of the second wavelength.

For example, consider the case where two fluorescent substances are present in the area of interest illuminated by the excitation radiation, and the radiation emitted by a first substance, in response to the excitation radiation, exhibits a first spectrum with a maximum at a first wavelength, while the radiation emitted by a second substance, in response to the excitation radiation, exhibits a second spectrum with a maximum at a second wavelength, the maxima of the first and second spectra being well separated (e.g., they are separated by at least 30 nm, or even by at least 50 nm). In this case, by filtering the radiation emitted by these substances, it is possible to emphasize the emission of one of the substances on a portion of the spectrum by reducing, on one of the images, the intensity of the signal corresponding to the fluorescence radiation detected on this portion of the spectrum (of course, it is also possible to do the opposite, namely to favor on the part of the spectrum, the emission of one of the substances by increasing, on one of the images, the intensity of the signal corresponding to the fluorescence radiation detected on this part of the spectrum).

For example, in one embodiment of this method, the filtering operation is carried out using an operating procedure in which a low-pass filter chosen to reduce the contribution of the signal emitted by the first substance is placed between the area of interest and the detector (regardless of the position of this filter in the optical path; it can be before or after the other filters or lenses) and an operating procedure in which this low-pass filter is removed.

Advantageously, the filter used for said filtering operation (in this example, a low-pass filter) is a removable filter. In this document, the adjective “removable” means that the filter can be easily put in place on the optical path between the area of interest and the detector or removed from this optical path, that is to say, without having to use a tool, for example. The same is true for the adjective “retractable”, the “retracted” position of the filter corresponding to a position that is not on the optical path between the area of interest and the detector. Similarly, an “adjustable” filter is considered in this document as a “removable” filter since its filtering function can easily be activated or removed. Thus, for example, the filtering operation makes it possible to reduce the contribution of the signal, which, without filtering, would mask the less intense signal. For this filtering operation to be useful, the substances whose fluorescence we wish to observe must have spectrally shifted maxima so that we can more specifically filter the signal corresponding to one of these maxima in order to identify another. The observed substances must, therefore, have respective responses (in particular in re-emission and without the filtering operation mentioned above) to the excitation radiation, which differs by intensity maxima spectrally shifted relative to each other over the detected wavelength range. Thus, thanks to the method according to the invention, it is possible to visualize at least two distinct images of the fluorescence radiation emitted in response to the same excitation radiation by fluorescent substances present in the area of interest illuminated by this excitation radiation.

For example, if the first substance corresponds to indocyanine green and the second substance is the one responsible for the autofluorescence of the parathyroid glands, with an excitation wavelength at 680 nm, we observe for indocyanine green a first maximum of the fluorescence emission spectrum at a first wavelength close to 820 nm, whereas with this same excitation wavelength, we observe for autofluorescence a second maximum of the fluorescence emission spectrum at a second wavelength close to 711 nm (see FIG. 4). To obtain an image in which the parathyroid glands are more visible than the tissues labeled with indocyanine green, a low-pass filter with a cut-off wavelength between 750 and 800 nm can be placed in front of the sensor (see FIG. 8). By removing this low-pass filter, the tissues marked with indocyanine green become more visible again or even mask the autofluorescence because they are much more fluorescent. We can, therefore, obtain a first image in which, for fluorescence radiation emitted with a wavelength greater than the cut-off wavelength, the ratio between the intensity of the emitted radiation and the intensity of the output signal is different from that in the second image. In the first image, this ratio is lower than in the second image.

The method according to the invention also optionally comprises one and/or the other of the following characteristics, each considered independently of one another or in combination with one or more others:
at least one displayed image corresponds essentially to the contribution, in the output signal, of the emission of the fluorescence radiation of the first substance or to the contribution, in the output signal, of the emission of the fluorescence radiation of the second substance, and is calculated from a combination of output signals obtained respectively using the first operating procedure for filtering and the second operating procedure for filtering,
it comprises the alternate production of at least one image of at least one parathyroid gland and at least one image of a vascular network supplying blood to this parathyroid gland,
the image of the parathyroid gland is made with a low-pass filter in place between the area of interest and the sensor, and the image of the vascular network is made without the low-pass filter between the area of interest and the sensor,
the image of the parathyroid gland is performed with a low-pass filter in place between the area of interest and the sensor, and the vascular network is imaged with a long-pass filter in addition to or instead of the low-pass filter between the area of interest and the sensor,
more generally, for example, in the first operation procedure for filtering, a band-pass filter is placed between the area of interest and the detector, and in the second operating procedure for filtering, this band-pass filter is removed,
for the excitation wavelength used to illuminate the area of interest, the fluorescence yield of the first substance is greater than the fluorescence yield of the second substance;
the excitation operation is carried out with a single excitation wavelength; for example, the excitation wavelength is chosen so as to favor the fluorescence re-emission function by the second substance compared to that of the first substance;
the operation of generating at least two images from the output signal comprises at least one different optical or digital processing for each of these two images;
the optical processing of one of said at least two images comprises the installation of a removable filter between the area of interest and the sensor, while the optical processing of the other of said at least two images comprises the removal of the removable filter from the optical path between the area of interest and the sensor; this removal may optionally be accompanied by the installation of a long-pass filter;
the digital processing comprises an operation consisting of using the signals respectively obtained on different color channels to generate each of said at least two images;
said at least two fluorescent substances are respectively indocyanine green and the substance responsible for the autofluorescence of the parathyroid glands;
the excitation radiation has a maximum intensity between 600 and 720 nm, for example, between 650 and 700 nm
the excitation radiation has a maximum intensity at 680 nm;
the detection and acquisition operation comprises
   an acquisition operation according to the first operating procedure, with the source producing the excitation radiation switched off,
   an acquisition operation according to the first operating procedure, with the source producing the excitation radiation switched on,
   an acquisition operation according to the second operating procedure, with the source producing the excitation radiation switched off,
   an acquisition operation according to the second operating procedure, with the source producing the excitation radiation switched on,
and operations consisting of calculating and displaying images of the contribution, in the output signal, of the emission of the fluorescence radiation of the first substance or of the contribution, in the output signal, of the emission of the fluorescence radiation of the second substance;
the operation of displaying images formed from the output signal comprises the display of an image representative of the coefficient α on at least a portion of the area of interest, where

$$α = \frac{( Low - bckLow )}{( High - bckHigh )}$$

With
Low=an image of the fluorescence radiation of the area of interest with the band-pass filter and the source producing the excitation radiation switched on,
bckLow=a background image with the band-pass filter and the source producing the excitation radiation switched off,
High=an image of the fluorescence radiation from the area of interest without the band-pass filter and with the source producing the excitation radiation switched on,
bckHigh=a background image without the band-pass filter, and the source producing the excitation radiation switched off.
   α corresponds to the proportion of the total autofluorescence signal from at least one parathyroid gland in the output signal after filtering by the band-pass filter.

The invention also relates to a fluorescence imaging device, suitable for example for implementing the method mentioned above. It includes at least one sensor, one filter, and one excitation light source.

For example, this device comprises an excitation light source configured to emit, on an area of interest, excitation radiation of autofluorescence or fluorescence. This excitation radiation is, for example, emitted in the form of a laser beam. This excitation radiation corresponds to an excitation wavelength defined by its maximum intensity on a spectrum. This excitation wavelength is, for example, included in a range of emission wavelengths between 600 nm and 850 nm.

This device also includes a detector configured to detect fluorescence radiation. For example, the detector is a CMOS or CCD camera comprising a sensor configured to detect fluorescence radiation emitted by at least a portion of the area of interest within a detection range that may extend over (or be included in) a detection wavelength band extending at least between 400 nm and 1000 nm. The sensor is also configured to generate at least one output signal whose intensity as a function of the fluorescence radiation wavelength is representative of the intensity of the fluorescence radiation detected by the sensor.

This device further includes filtering means. The filtering means are, in particular, configured to be able to implement at least two different operating procedures and to generate from the output signal at least two images, each respectively using one of these different operating procedure for filtering, the respective contribution to the intensity of the output signal obtained respectively at a first wavelength and a second wavelength distinct from the detection page, being different with each of these two filtering operating procedures. For example, with one of the two operating procedures, the contribution to the output signal strength obtained at the level of the second wavelength is greater than or equal to that obtained at the level of the first wavelength; with the other operating procedure, conversely, the contribution to the output signal strength obtained at the level of the first wavelength is greater than or equal to that obtained at the level of the second wavelength. For example, the first and second methods of filtering are implemented one after the other; the first operating procedure for filtering promoting, when first and second substances are present in the area of interest, the contribution of the emission of fluorescence radiation of the second substance in the output signal, relative to the contribution of the emission of fluorescence radiation of the first substance, and the second operating procedure for filtering promoting the contribution of the emission of fluorescence radiation of the first substance in the output signal, relative to the contribution of the emission of fluorescence radiation of the first substance in the output signal obtained with the first operating procedure for filtering.

Furthermore, the device comprises calculation means configured to carry out operations consisting of calculating images obtained either, on the one hand, from the output signal obtained using the first operating procedure for filtering and, on the other hand, from the output signal obtained using the second operating procedure for filtering, or from a combination of output signals obtained respectively using the first filtering operating mode and the second operating procedure for filtering.

This device also includes display means for displaying images formed from the output signal.

This device may also include one and/or more of the following characteristics, each considered independently of the next or in combination with one or more others:
the excitation wavelength is included in a range of emission wavelengths between 650 nm and 700 nm,
the filtering means are configured so that with one of the two operating procedures, the contribution to the output signal intensity obtained at the level of the second wavelength is greater than that obtained at the level of the first wavelength, and with the other operating procedure, conversely, the contribution to the output signal intensity obtained at the level of the first wavelength is greater than that obtained at the level of the second wavelength,
the filtering means comprise a removable filter; this removable filter is, for example, a low-pass filter having a cut-off wavelength between 720 nm and 800 nm or between 720 nm and 800 nm;
the removable filter is placed between the area of interest and the sensor in the first operating procedure and is removed in the second operating procedure,
it is configured to alternately produce at least one image of at least one parathyroid gland with the first operating procedure and at least one image of the vascular network supplying blood to this parathyroid gland, which is irrigated with indocyanine green, with the second operating procedure, the device also being configured to receive a low-pass filter in the optical path located between the area of interest and the sensor, in the first operating procedure and to remove, in the second operating procedure, this low-pass filter from the optical path,
the low-pass filter has a cut-off wavelength adapted to limit the indocyanine green fluorescence emitted in the area of interest
it comprises a protective cover for a camera comprising the sensor and in which the filter is arranged on the cover or integrated into the cover,
the output signal comprises the signal of at least two different color channels. This device comprises means for processing the output signal configured to produce linear combinations of the intensities obtained on the different color channels.

According to another aspect, the invention relates to a computer program comprising program code instructions for executing the above-mentioned method when said program is executed on a computer.

In this document, the operations described as being performed on a signal or on an image correspond to operations on quantities representative of this signal (for example, the signal generated at the level of a photosensitive element of a sensor) or values associated with at least certain pixels of the image. Non-descript signal processing may also be applied. For example, operations such as summation, registration, normalization, colorization, etc., which are well-known to those skilled in the art, may be carried out in addition to those described below.

### BRIEF DESCRIPTION OF THE DRAWINGS

Other characteristics and advantages will become apparent in the detailed description of different embodiments of the invention, the description being accompanied by examples and references to the accompanying drawings.

FIG. 1 is a schematic view of an exemplary embodiment of an imaging device according to the invention;

FIG. 2 shows the transmission characteristics of a long-pass filter as a function of the wavelength used in an exemplary implementation of the method according to the invention;

FIG. 3 is a schematic representation of a filter system used in an exemplary implementation of the method according to the invention;

FIG. 4 is a normalized representation of the emission spectra of parathyroid gland autofluorescence and indocyanine green as seen by the camera for excitation at 680 nm (without the removable low-pass filter, but with other filters such as a Bayer filter and a long-pass filter);

FIG. 5 shows the spectral characteristics of an example of a CMOS sensor used in an example of implementation of the method according to the invention;

FIG. 6 shows an image of an area of interest obtained by positioning a low-pass filter between this area of interest and the sensor of the device according to the invention; we essentially observe the autofluorescence of a parathyroid gland;

FIG. 7 shows an image equivalent to that of FIG. 6 but obtained without the removable low-pass filter; we mainly observe the fluorescence of indocyanine green;

FIG. 8 is a normalized representation of the emission spectra of parathyroid gland autofluorescence and indocyanine green as seen by the camera for excitation at 680 nm (without the removable low-pass filter, but with other filters such as a Bayer filter and a long-pass filter);

### DETAILED DESCRIPTION OF THE EMBODIMENTS

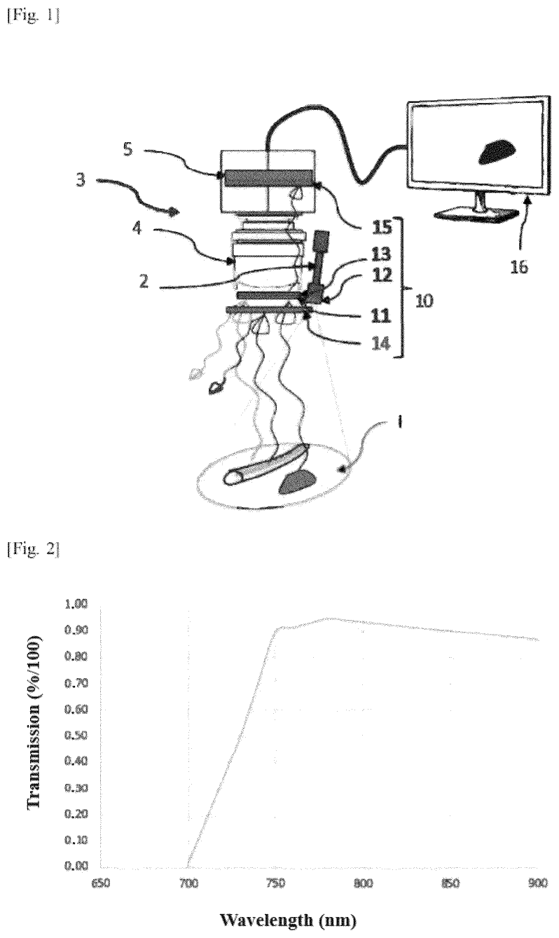

An example of an embodiment of a device 1 according to the invention is shown in FIG. 1, comprising an excitation light source 2, a detector 3 with an objective 4 comprising at least one optical lens. It also includes a filtering means 10. (Note that the presence of a Bayer filter in the filtering means is optional).

Device 1 also comprises calculation means (one or more computers or servers) configured to perform various operations from the output signal or signals of the detector 3.

The excitation light source 2 is, for example, a laser source. This laser source emits excitation radiation with a maximum intensity corresponding to an excitation wavelength of, for example, between 600 nm and 800 nm or, for example, between 650 and 720 nm. More specifically, the excitation wavelength is 680 nm.

Detector 3 is, for example, a camera equipped with a 5 CMOS or CCD sensor. This camera is, for example, a CMV 2000-type model marketed by XIMEA.

According to a first embodiment of the filtering means 10, these comprise, for example, a lighting filter 11, an excitation light source filter 12, a long-pass or band-pass filter 13 and a low-pass filter and/or a long-pass filter 14, advantageously removable. It should be noted that while a 13 long-pass filter may be sufficient in certain situations, in other situations, and in particular if powerful ambient lighting is used, it will be preferable to use a band-pass filter with a cut-off frequency above 900 nm (see Document EP2840953A1). The filtering means 10 may also include a matrix of color filters 15 (e.g., a Bayer filter mosaic) placed in front of the detector 3. (As indicated above, the presence of this type of filter in the filtering means is optional).

The lighting filter 11 is placed between the area of interest I, which includes the fluorescent or autofluorescent tissues, and the sensor 5. The illumination filter 11 is used to filter white light produced by light-emitting diodes fitted to the detector 3 and/or the operating light (e.g., for example, produced by a scialytic).

The excitation light source filter 12 is placed downstream of the excitation light source 2, between the latter and the area of interest I. For example, the excitation light source filter 12 is a band-pass filter that passes the excitation radiation emitted by the excitation light source 2. The excitation light source filter 12 essentially allows excitation radiation to pass, for example, over more or less 10 nm around the excitation wavelength, and blocks radiation outside this range.

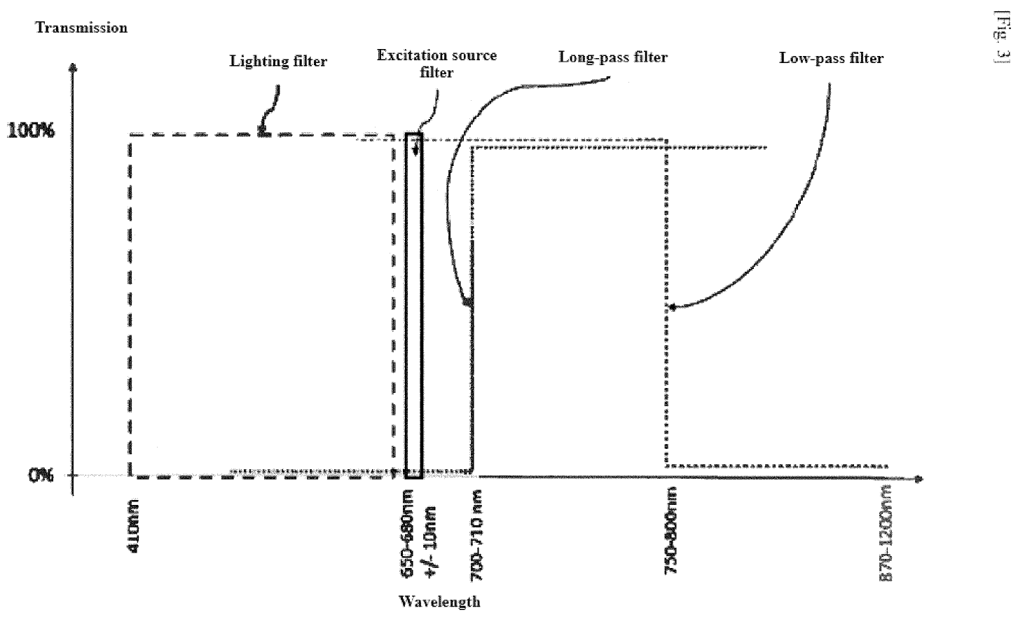

The long-pass or band-pass filter 13 is placed between an area that includes the fluorescent or autofluorescent tissues of the area of interest I and the detector 3. For example, the long-pass filter 13 is placed in front of the objective 4. The long-pass or band-pass filter 13 has a cut-off wavelength located above the excitation wavelength. For example, this long-pass filter 13 has a spectral response such as that shown in FIG. 2, with a cut-off wavelength between 700 and 750 nm. In this example, the long-pass filter transmits more than 80% of the light between the cut-off wavelength and at least up to 900 nm. For example, the low-pass filter 14 has a cut-off wavelength that lies between the wavelengths of the intensity maxima of the fluorescence emission spectrum of two substances to be differentially imaged at a particular excitation wavelength. In particular, the cut-off wavelength is chosen to detect the substance having the maximum intensity at the lowest wavelength when the low-pass filter is in place and to detect the substance having the maximum intensity at the highest wavelength when the low-pass filter is removed.

The low-pass filter 14 is removable and can be placed between the area that includes the fluorescent or autofluorescent tissues and the detector 3. For example, the low-pass filter 14 is placed in front of the objective 4. The low-pass filter 14 has a cut-off wavelength located above the excitation wavelength (and above the cut-off wavelength of the long-pass filter 13). For example, this low-pass filter 14 has a cut-off wavelength between 750 and 800 nm. For example, this low-pass filter 14 has a cut-off wavelength of around 775 nm.

The low-pass filter 14, and/or possibly a long-pass filter, can be placed in different configurations depending on the solution adopted. For example, according to a “retractable” or “shutter” type solution, the low-pass filter 14 is placed on a filter wheel or a shutter (advantageously, this shutter is integrated into the camera) so as to be able to be retracted or removed, or on the contrary placed on the optical path. Another solution may be provided by an “adjustable” filter, such as an activatable liquid crystal filter, or one with an adjustable or tunable cut-off wavelength, as is the case with VersaChrome Edge™ filters, marketed by the Semrock company, which allow the cut-off wavelength to be moved between 770 nm and 900 nm, for example.

In an alternative solution, the low-pass filter 14 is placed on or in a sterile housing designed to protect the camera and its objective 4. For example, very simple filters with a cut-off power of 1.OD to 2.OD can be used. Such filters can easily be added to an existing fluorescence detection device. They just serve to significantly reduce a portion of the signal. They can be made from very low cost filters or plastic films sold in large widths to protect from the sun.

For example, the sterile cover intended to protect the camera and its objective 4 is supplied with one or more sterile filters of this type, which can be arranged on the front surface of the cover. The filter can be attached to the housing using a notch, a self-adhesive strip, a Velcro®-type strip, a magnetic fastener integrated into the cover, etc. Thus, during surgery and depending on its progress, the surgeon can put on or remove a filter before using the camera.

FIG. 3 schematically illustrates an example of filtering as a function of the wavelength by the different filters of the filtering means 10 described previously.

The filtering means 10 are adapted to be able to occupy at least two different configurations. According to a first configuration, the low-pass filter 14 is placed between the area of interest I and the detector 3. According to a second configuration, the low-pass filter 14 is retracted or replaced by a long-pass or band-pass filter.

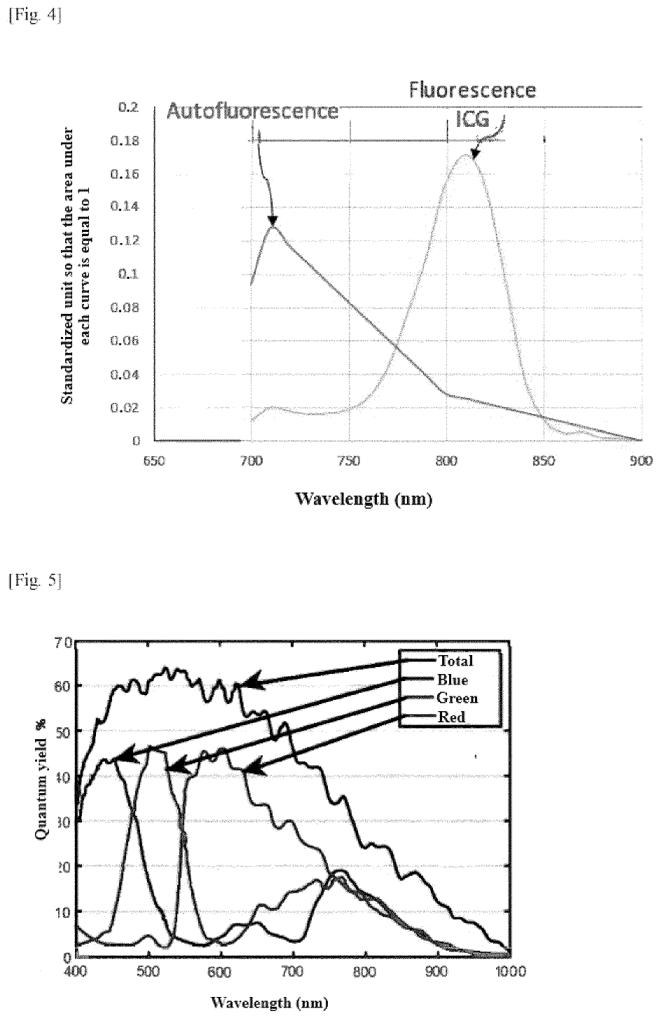

FIG. 4 shows the emission spectra corresponding to the parathyroid gland autofluorescence and indocyanine green fluorescence, respectively, when the excitation light source has an excitation wavelength of 680 nm.

Using the device according to the invention described above, it is possible to implement the method according to the invention in several ways.

According to a first embodiment of the method according to the invention, a filtering operation is carried out according to a first and a second operating procedure for filtering, which are different from each other and implemented one after the other. The first operating procedure for filtering is implemented using a low-pass filter placed in its first configuration (i.e., placed between the area of interest I and the detector 3). This low-pass filter has a cut-off wavelength of less than 800 nm (e.g., 775 nm). Detector 3 can then detect 60% of the autofluorescence signal and 10% of the indocyanine green signal. So, with a low-pass filter 14 of this type, the detected radiation corresponds essentially to the autofluorescence signal of the parathyroid glands. As illustrated in FIG. 6, the image then generated for display on the display means 16 essentially shows the autofluorescence of one or more parathyroid glands.

To further reduce the contribution of indocyanine green, a lower cut-off wavelength would have to be used for the low-pass filter 14, but this would be to the detriment of the quality of the measured autofluorescence signal. Indeed, detector 3 would detect less signal from indocyanine green fluorescence but also less of the autofluorescence signal because the detection will be done over a narrower bandwidth in which the measured intensity of the autofluorescence signal is low or even very low.

On the other hand, if the low-pass filter 14 is in its second configuration (retracted), which corresponds to the second operating procedure for filtering, and the indocyanine green has not yet been injected, the parathyroid glands are very visible. In fact, the parathyroid glands are then detected over a very large portion of their emission spectrum, from 700 nm to 900 nm. Furthermore, with an excitation wavelength around 650-700 nm, the intensity of the autofluorescence is greater than with an excitation wavelength between 700 and 800 nm, the parathyroid glands are, therefore, even more visible than with a device of the prior art using an excitation wavelength between 750 nm and 800 nm, for example.

The autofluorescence signal of the parathyroid glands can still be observed, while the indocyanine green has diffused little. But, when the perfusion of indocyanine green is complete, detector 3 detects both the autofluorescence signal and the fluorescence signal of indocyanine green, but since the latter is more intense, the signal from the parathyroid glands is masked (see FIG. 7).

Furthermore, by positioning, between the area of interest I and detector 3 (first configuration), a low-pass filter 14, which has a cut-off wavelength between 700 and 900 nm, or a long-pass filter which has a cut-off wavelength between 750 and 800 nm, it is possible to adjust the ratio of the respective signals of autofluorescence and indocyanine green fluorescence to produce a hybrid image (possibly with a similar or identical fluorescence level for each of the signals).

At least four types of images can potentially be obtained with device 1 described above.
1) an image over a low wavelength range (below the cut-off of the low-pass filter 14) with the low-pass filter placed between the area of interest I and detector 3 (first configuration/first operating procedure),
2) an image over a detection range between 700 and 1000 nm, without the low-pass filter 14 (second configuration/second operating procedure),
3) an image over a high wavelength range, replacing the low-pass filter 14 with a long-pass filter (third operating procedure),
4) a combined image if the low-pass filter 14 has a cut-off wavelength between 780 and 800 nm (fourth operating procedure).

According to a second embodiment of the filtering means 10, these comprise, for example, a lighting filter 11, an excitation light source filter 12, and a long-pass or band-pass filter 13, such as those described above in relation to the first embodiment of the filtering means 10.

The filtering means 10, optionally further comprise a matrix of color filters 15 (for example a Bayer filter mosaic) placed in front of the detector 3 (As indicated above, the presence of this type of filter in the filtering means is optional).

Thus, the filtering means 10 are configured to collect signals in different wavelength ranges depending on the color channel(s) used for processing the fluorescence images.

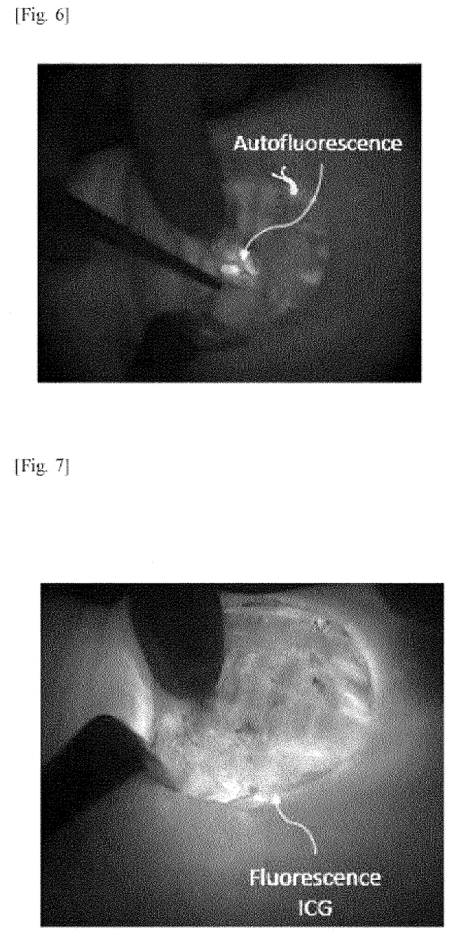

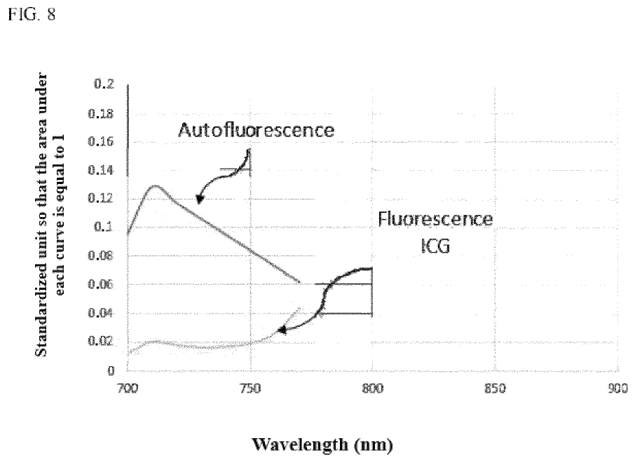

Indeed, as shown in FIG. 5, the spectral response of the detector 3 equipped with the color filter matrix 15 can differ depending on the colors, in particular in a spectral band between 700 and 900 nm. Therefore, by using an appropriate color filter matrix 15, such as that giving the spectral response illustrated in FIG. 5, it is possible to carry out digital filtering by considering certain channels and/or combining them. For example, by processing with appropriate signal processing means the signals obtained from the red and blue channels (second operating procedure for filtering), it is possible to detect, measure, and visualize both the autofluorescence signal and the fluorescence signal. By processing the signals obtained from the blue channel, it is possible to detect, measure, and visualize essentially the fluorescence signal. By subtracting the intensity of the signal obtained on the blue channel from that of the signal obtained on the red channel, it is possible to detect, measure, and visualize essentially the autofluorescence signal (first operating procedure for filtering). Other combinations are possible. By adding to the intensity of the signal obtained on the red channel twice the intensity of the signal obtained on the green channel and subtracting three times the intensity of the signal obtained on the blue channel, it is possible to detect, measure, and visualize essentially the autofluorescence signal.

Color filter matrices 15 other than a Bayer filter mosaic may optionally be used. For example, to promote detection on one or more other wavelength ranges, we can use a CYGM filter (cyan, yellow, green, magenta), an RGBE filter (red, green, blue, emerald), a CMYK filter (cyan, magenta, yellow and white), an RGBW filter (red, green, blue, white), etc. Similarly, rather than a mosaic of filters, one can use three separate sensors (e.g., three separate CCDs) or even superimposed filters, as in a Foveon X3 sensor, etc. This list of example filters and sensors is not exhaustive. In any case, they can be used to select one or more channels and/or obtain various combinations of the signals obtained on different channels.

It is also possible to combine the use of a removable filter (low-pass and/or long-pass) with the filter matrices.

According to a third embodiment of the method according to the invention, this makes it possible to completely separate the respective contributions of indocyanine green and autofluorescence in the emission of fluorescence radiation by the area of interest I. Furthermore, this third embodiment of the method, according to the invention also makes it possible to measure the increase in the contribution of indocyanine green between two successive times, for example, to measure the vascularization of a parathyroid gland.

In order to determine the respective contributions of indocyanine green and autofluorescence in the fluorescence radiation collected by sensor 5, it is assumed that this radiation only includes contributions from indocyanine green, autofluorescence, and little contribution related to ambient light, which always contains some infrared in the detection band of device 1. Other potential contributions to the signal are neglected.

It is also assumed that the proportion of signal emitted as autofluorescence or fluorescence by indocyanine green and detected according to the different measurement configurations is constant regardless of the concentration of fluorescent material. It is known that, for example, for indocyanine green, the emission spectrum depends partly on the concentration and that at very high concentrations a “quenching” phenomenon (extinction of the signal) can be observed. However, this phenomenon is totally negligible in relation to the allowable injectable doses of indocyanine green.

Then, according to the third embodiment of the method according to the invention, the following acquisitions are carried out:
1) With the low-pass filter 14 (IR-cut) placed between the area of interest and the sensor 5:
An acquisition (1A) with the excitation light source 2 (laser) off,
An acquisition (1B) with the excitation light source 2 on.
2) With the low-pass filter 14 (IR-cut) retracted (removed from the optical path):
An acquisition (2A) with the excitation light source 2 off,
An acquisition (2B) with the excitation light source 2 on.

This series of acquisitions provide an image that contains only the contribution of the indocyanine green contained in the tissue to the fluorescence emission collected by sensor 5 and an image that contains only the contribution of the autofluorescence of the tissue observed (parathyroid glands) to the fluorescence emission collected by sensor 5.

Acquisition (1A) gives a background image with filter 14. This image is named bckLow.

Acquisition (1B) gives an image of the fluorescence radiation of the area of interest with filter 14. This image is named Low.

$$Low = bckLow + ( α * AF + β * ICG ) * δ / {d}^{2}$$

Acquisition (1A) gives a background image with filter 14. This image is named bckHigh.

The acquisition (2B) gives an image of the fluorescence radiation from the area of interest without the filter 14. This image is named High.

$$High = bckHigh + ( AF + ICG ) * δ / {d}^{2}$$

Where
AF corresponds to the total autofluorescence signal detected with device 1 without filter 14.
ICG corresponds to the total indocyanine green fluorescence signal detected with device 1 without filter 14.
d corresponds to the distance between the excitation source 2 and the area of interest I
α corresponds to the proportion of the total autofluorescence signal detected with device 1, filtered by low-pass filter 14.
β corresponding to the proportion of the total indocyanine green fluorescence signal detected with device 1, filtered by low-pass filter 14.
δ is the gain of detector 3. It has been verified that this gain is similar for the fluorescence of indocyanine green and for the autofluorescence of a parathyroid gland.

Calculation of an indocyanine green image and an autofluorescence image.

From the previous equations, we obtain:

$$( Low - bckLow ) * \frac{{d}^{2}}{δ} = α * A F + β * ICG$$

$$( High - bckHigh ) * \frac{{d}^{2}}{δ} = AF + ICG$$

The combination

$$( Low - bckLow ) - α * ( High - bckHigh ) = ( β - α ) * \frac{δ}{{d}^{2}} * IC$$

provides the image of the contribution of indocyanine green fluorescence to within a multiplicative factor varying with distance.

The combination

$$( Low - bckLow ) - β * ( High - bckHigh ) = ( α - β ) * \frac{δ}{{d}^{2}} * AF$$

provides the image of the contribution of the autofluorescence of the parathyroid glands to a multiplicative factor varying with the distance.

It is, therefore, possible to visualize the contribution of autofluorescence alone and of indocyanine green fluorescence alone to within a multiplicative factor.

If the measurements are taken at a constant distance, for example, using a wedge, the values can be compared between different surgeries. It is also possible to use a device that measures the distance using a pointer of the type described in patent application FR1750361A filed by Fluoptics® or of the type of the FLUOBEAM®LS device. Measuring the distance allows you not to worry about the position of the probe at the time of measurement. This also makes it possible to obtain an absolute value of the signal level emitted at the level of the area of interest I.

Analysis of perfusion after injection of indocyanine green.

The same type of measurement can be used to quantify the signal increase during perfusion analysis following an injection of indocyanine green.

By taking up the previous notations, by indexing by 1 the acquisition series at a first-time t and by 2 the acquisition series at a second-time t′ subsequent to t, and by using the fact that the parathyroid is the same and has an identical autofluorescence radiation at times t and t′ (just before the injection and after) we have

$$( {Low}_{1} - {bckLow}_{1} ) * \frac{{d}_{1}^{2}}{δ} = α * AF + β * {ICG}_{1}$$

$$( {High}_{1} - {bckHigh}_{1} ) * \frac{{d}_{1}^{2}}{δ} = AF + {ICG}_{1}$$

$$and$$

$$( {Low}_{2} - {bckLow}_{2} ) * \frac{{d}_{2}^{2}}{δ} = α * AF + β * {ICG}_{2}$$

$$( {High}_{2} - {bckHigh}_{2} ) * \frac{{d}_{2}^{2}}{δ} = AF + {ICG}_{2}$$

Which allows the following to be obtained

$$\frac{( {Low}_{1} - {bckLow}_{1} )}{( {High}_{1} - {bckHigh}_{1} )} = \frac{α * AF + β * {ICG}_{1}}{( AF + {ICG}_{1} )}$$

$$or$$

$$( AF + {ICG}_{1} ) * ( {Low}_{1} - {bckLow}_{1} ) = ( α * AF + β * {ICG}_{1} ) * ( {High}_{1} - {bckHigh}_{1} )$$

$$and$$

$$AF * ( {Low}_{1} - {bckLow}_{1} - α * ( {High}_{1} - {bckHigh}_{1} ) ) = {ICG}_{1} * ( β * ( {High}_{1} - {bckHigh}_{1} ) - ( {Low}_{1} - {bckLow}_{1} ) )$$

From where

$$\frac{{ICG}_{1}}{AF} = \frac{( {Low}_{1} - {bckLow}_{1} - α * ( {High}_{1} - {bckHigh}_{1} ) )}{( β * ( {High}_{1} - {bckHigh}_{1} ) - ( {Low}_{1} - {bckLow}_{1} ) )}$$

By doing the same reasoning for the acquisition at the second time t′, we obtain

$$\frac{{ICG}_{2}}{AF} = \frac{( {Low}_{2} - {bckLow}_{2} - α * ( {High}_{2} - {bckHigh}_{2} ) )}{( β * ( {High}_{2} - {bckHigh}_{2} ) - ( {Low}_{2} - {bckLow}_{2} ) )}$$

We can, therefore, provide distance-independent confidence values that allow comparisons
either between the concentration levels of indocyanine green between two measurement times (two data acquisitions using device 1).

$$\frac{{ICG}_{2}}{{ICG}_{1}} = \frac{\frac{( {Low}_{2} - {bckLow}_{2} - α * ( {High}_{2} - {bckHigh}_{2} ) )}{( β * ( {High}_{2} - {bckHigh}_{2} ) - ( {Low}_{2} - {bckLow}_{2} ) )}}{\frac{( {Low}_{1} - {bckLow}_{1} - α * ( {High}_{1} - {bckHigh}_{1} ) )}{( β * ( {High}_{1} - {bckHigh}_{1} ) - ( {Low}_{1} - {bckLow}_{1} ) )}}$$

or between different surgeries, by a comparison of the increase in the indocyanine green signal compared to the autofluorescence of the parathyroid glands.

$$\frac{( {ICG}_{2} - {ICG}_{1} )}{AF} = \frac{( {Low}_{2} - {bckLow}_{2} - α * ( {High}_{2} - {bckHigh}_{2} ) )}{( β * ( {High}_{2} - {bckHigh}_{2} ) - ( {Low}_{2} - {bckLow}_{2} ) )} - \frac{( {Low}_{1} - {bckLow}_{1} - α * ( {High}_{1} - {bckHigh}_{1} ) )}{( β * ( {High}_{1} - {bckHigh}_{1} ) - ( {Low}_{1} - {bckLow}_{1} ) )}$$

So, this method only requires knowing in advance the two parameters α and β. However, these values can be measured by calibration.

### Calibration

The purpose of calibration is to estimate the proportion α of the autofluorescence signal and the proportion β of indocyanine green fluorescence detected by device 1 in the different measurement configurations and the different operating procedures for filtering.

For this purpose, it is possible to determine these measurements prior to the acquisition of fluorescence images in the different measurement configurations and the different operating procedures for filtering. It is possible to determine these values once for all subsequent uses of device 1 or even for all devices 1 used, meeting the same technical specifications (same type of sensor 5, same filters, etc.). Indeed, it could be verified that these coefficients α and β are relatively independent of the conditions of acquisition of the fluorescence signal by the sensor 5.

Thus, an acquisition is carried out in total darkness, with only autofluorescence emission by the parathyroid and an acquisition is carried out in total darkness, with only fluorescence emission by indocyanine green.

We, therefore, obtain two series of measurements:

For autofluorescence alone

$$( Low - bckLow ) * \frac{{d}^{2}}{δ} = α * AF$$

$$( High - bckHigh ) * \frac{{d}^{2}}{δ} = AF$$

Therefore, the value of α can be determined from filtered and unfiltered images acquired with only autofluorescence emission from the parathyroid

$$α = \frac{( Low - bckLow )}{( High - bckHigh )}$$

It can be noted that the value of α depends on the type of fluorescence emission. Thus, the value of α is not the same for parathyroid autofluorescence as for fluorescence or autofluorescence of other tissues or substances. By producing one (or more) images from the values of α (on an area or the entire region of interest I, it is possible to detect false positives or even pathologies of the parathyroid).

For indocyanine green fluorescence alone

$$( Low - bckLow ) * \frac{{d}^{2}}{δ} = β * ICG$$

$$( High - bckHigh ) * \frac{{d}^{2}}{δ} = ICG$$

Therefore, the value of β can be determined from the filtered and unfiltered images acquired with only the indocyanine green emission alone.

$$β = \frac{( Low - bckLow )}{( High - bckHigh )}$$

In any case, this calibration would have to be carried out for all the pixels of sensor 5. However, pixel responses can be assumed to vary according to the black value and gain of each pixel. We can also consider that the spectral response of the pixels of sensor 5 is identical for all pixels. It follows from these hypotheses that it is sufficient to determine α and β on an area of the sensor 5 and that the values thus determined will be the same for all the pixels.

To monitor the evolution of the indocyanine green perfusion, we chose to use the ratio

$$\frac{( {ICG}_{2} - {ICG}_{1} )}{AF} .$$

However, it is possible to use other quantities as a basis. For example, the report

$$\frac{{ICG}_{1}}{{ICG}_{2}}$$

may be preferable to

$$\frac{{ICG}_{2}}{{ICG}_{1}} ,$$

if there is only one injection and this takes place only at the end of the surgery.

For quantitative measurement of indocyanine green and parathyroid autofluorescence values, it is necessary to work at a constant distance (which is possible by using a wedge, as already indicated above). If the different acquisitions (with light source 2 on or off and changing the operating procedure for filtering) are carried out sufficiently quickly, we can also consider that the distance has not varied significantly between these different acquisitions.

This disclosure includes electronics, electronic instructions, processors, memory, displays, and other means necessary to guide, control, display, and record the systems and processes described herein.

This disclosure includes imaging systems and equipment for various medical uses, including, but not limited to, imaging of thyroid and parathyroid tissues. The disclosure also includes methods of operating imaging systems and equipment, methods of differential imaging of human tissues for medical purposes, and methods of imaging parathyroid and thyroid tissues.

1.-30. (canceled)

31. A method of alternately imaging a parathyroid gland and a vascular network supplying blood to the parathyroid gland, the parathyroid gland and the vascular network being in a neck region of a patient, the method comprising:
providing a fluorescent imaging arrangement, the fluorescent imaging arrangement comprising:
a sensor, the sensor being configured to detect fluorescent radiation;
an excitation light source;
a first filter, the first filter being configured to be movable for selectively filtering light reaching the sensor, the first filter having a lower cut-off wavelength in a range of 750-800 nm; and
a display;
generating an indocyanine green (ICG) fluorescence image of the vascular network by:
providing ICG to the vascular network supplying blood to the parathyroid gland;
providing excitation light to the neck region, the excitation light having a maximum intensity between 650-700 nm;
using the sensor, detecting radiation comprising fluorescence radiation from ICG in the vascular network, and generating an ICG output signal;
generating an ICG fluorescence image of the neck region and displaying the ICG fluorescence image on the display, the ICG fluorescence image showing ICG fluorescence in said vascular network supplying blood to the parathyroid gland;
generating an autofluorescence parathyroid image after said generating an indocyanine green (ICG) fluorescence image, with at least some of said ICG still present in the vascular network, by:
positioning the first filter so that it filters light passing from the neck region to the sensor;
providing the excitation light to the neck region, the excitation light having a maximum intensity between 650-700 nm;
using the sensor, detecting radiation comprising autofluorescence radiation from the parathyroid gland and generating an autofluorescence output signal, wherein the radiation is filtered by the first filter so as to favor the contribution of parathyroid autofluorescence over ICG fluorescence;
generating an autofluorescence parathyroid image of the neck region and displaying the autofluorescence parathyroid image on the display, the autofluorescence parathyroid image comprising autofluorescence light of the parathyroid gland for viewing the parathyroid gland while ICG remains present in the neck region.

32. The method of claim 31, wherein the first filter is a low-pass filter with a cut-off wavelength between 750-800 nm which blocks a greater proportion of ICG fluorescence radiation than parathyroid gland autofluorescence radiation.

33. The method of claim 31, wherein the excitation light source is a 680 nm laser.

34. The method of claim 31, further comprising before said generating an indocyanine green (ICG) fluorescence image of the vascular network:
generating and displaying a preliminary autofluorescence parathyroid image of the neck region, the preliminary autofluorescence image being generated before ICG is provided to the vascular network, and comprising autofluorescence light of the parathyroid gland for viewing the parathyroid gland on the display.

35. The method of claim 31:
wherein the indocyanine green (ICG) fluorescence image is generated in an ICG mode of the fluorescent imaging arrangement;
wherein the autofluorescence parathyroid image is generated in an autofluorescence mode of the fluorescent imaging arrangement; and
wherein the method comprises alternately generating images of the neck region in the autofluorescence mode, then in the ICG mode, and then again in the autofluorescence mode.

36. The method of claim 31:
wherein the indocyanine green (ICG) fluorescence image is generated in an ICG mode of the fluorescent imaging arrangement, the first filter being in an inactive position in the ICG mode;
wherein the autofluorescence parathyroid image is generated in an autofluorescence mode of the fluorescent imaging arrangement, the first filter in an active position, being positioned for filtering light traveling to the detector in the autofluorescence mode.

37. The method according to claim 36, wherein the method comprises:
an acquisition operation (1A) in autofluorescence mode, with the excitation light source switched off,
an acquisition operation (1B) in autofluorescence mode, with the excitation light source switched on,
an acquisition operation (2A) in the ICG mode, with the excitation light source switched off,
an acquisition operation (2B) in the ICG mode, with the excitation light source switched on, and
using input from a plurality of said acquisition operations: calculating and displaying images of a contribution of an output signal from the sensor of at least one of autofluorescence radiation from the parathyroid gland and ICG fluorescence in the vascular network supplying blood to the parathyroid gland.

38. The method according to claim 37, wherein the operation of displaying images formed from the output signal comprises displaying an image representative of the coefficient α of at least a portion of the area of interest (I) comprising the neck region, where
$$α = \frac{( Low - bckLow )}{( High - bckHigh )}$$
with
Low=an image of the area of interest with the first filter in the active position, and the excitation light source switched on,
bckLow=an image of the area of interest with the first filter in the active position, and the excitation light source switched off,
High=an image of the area of interest with the first filter in the inactive position, and the excitation light source switched on,
bckHigh=an image of the area of interest with the first filter in the inactive position, and the excitation light source switched off.

39. The method of claim 38, wherein the images Low, bckLow, High, and bckHigh are all taken of the same neck area of the patient.

40. The method of claim 38, wherein a corresponds to the proportion of the total autofluorescence signal of at least one parathyroid gland in the output signal after filtering by the first filter.

41. The method according to claim 31, wherein the method comprises displaying an image representative of the coefficient α of at least a portion of an area of interest (I) comprising the neck region, where
$$α = \frac{( Low - bckLow )}{( High - bckHigh )}$$
with
Low=an image of the area of interest with the first filter in the active position, and the excitation light source switched on,
bckLow=an image of the area of interest with the first filter in the active position, and the excitation light source switched off,
High=an image of the area of interest with the first filter in the inactive position, and the excitation light source switched on,
bckHigh=an image of the area of interest with the first filter in the inactive position, and the excitation light source switched off.
